(19) United States
(12) Patent Application Publication
(10) Pub. No.: US 2003/0134783 A1
(43) Pub. Date: Jul. 17, 2003

(54) Use of cyclic heptapeptides for the inhibition of biofilm formation

(76) Inventors: Rasika M. Harshey, Austin, TX (US); Joe R. Mireles, Austin, TX (US); Adam Toguchi, Austin, TX (US)

Correspondence Address: Gardere Wynne Sewell LLP, 3000 Thanksgiving Tower, 1601 Elm Street, Suite 3000, Dallas, TX, 75201-4767, US

(21) Appl. No.: 10/201,636

(22) Filed: Jul. 22, 2002

Related U.S. Application Data
(60) Provisional application No. 60/308,933, filed on Jul. 31, 2001.

(51) Int. Cl. A61L 2/00; B05D 3/00; A61K 38/17

(52) U.S. Cl. 514/9; 514/12; 427/2.24; 427/2.28

(57) ABSTRACT

The present invention includes a coating for medical and industrial objects and compositions for the coating. One form of the present invention is a method for applying the coating to the medical or industrial objects. Another form of the invention is the production of biofilm-resistant paint and plastics. The invention also includes a method of dispersing pre-formed biofilms.

This application claims priority to U.S. Provisional Patent Application Serial No. 60/308,933, filed Jul. 31, 2001.

The U.S. Government may own certain rights in this invention pursuant to the terms of the National Institute of Health Grant No. GM57400.

### FIELD OF THE INVENTION

The invention relates generally to antimicrobial agents and specifically, to the use of cyclic heptapeptides in the inhibition of biofilm formation.

### BACKGROUND OF THE INVENTION

Biofilms are matrix-enclosed bacterial populations adherent to each other and/or to surfaces or interfaces. Biofilms are difficult to dissipate because they are resistant to antimicrobial agents and detergent. Biofilms are medically important because they contaminate biologic surfaces, devices and instruments, including contact lenses, intrauterine devices, catheters, pacemakers, artificial limbs, joint implants, and they cause gum disease and tooth decay. Industrial problems caused by biofilm formation include corrosion of materials ranging from metals to concrete, problems in industrial water systems ranging from clogging of pipes to fouling of heat exchangers and corrosion of computer chips.

Removal of biofilm formation is generally accomplished by the use of antimicrobial agents. These antimicrobial agents are of varying chemical composition and can include surfactants, metal-based compositions, various polymers, and antibiotics. By definition, surfactants are amphipathic compounds able to stabilize suspensions of non-polar materials in aqueous solution. According to this definition, common surfactants are soap and household or industrial detergents. Biosurfactants are surfactants from living organisms. They are biodegradable, potentially less toxic than synthetic surfactants, and have structures and functions that are different from those of synthetic surfactants. The primary composition of most known surfactants are lipopeptides or glycolipids. One such lipopeptide, formed by Bacillus subtilis, is termed surfactin. Surfactin is a cyclic lipopeptide formed by a heptapeptide and a lipid portion constituted by a mixture of beta-hydroxy fatty acids with chains having between 13-15 carbon atoms.

The methods currently in use for prevention of biofilms act at the level of biofilm removal and, generally, do not interfere with the formation of the biofilm. These removal methods are costly, often involve the use of caustic chemicals, and provide only short-term prevention. In medical devices, various techniques have been described that incorporate potentially toxic metal ions in the form of metal salts into materials that make up the medical devices. The protection against biofilm formation lasts only as long as the coating remains on the surface of the device. Biofilms in water systems are generally removed by the addition of an antimicrobial agent, often a surfactant, to the water system. In this case, protection is dependent upon the stability of the compound so that continuous addition is required to prevent biofilm formation. Accordingly, a method of long-term prevention from biofilm formation is needed, one that acts to prevent biofilm formation rather than merely its removal.

### SUMMARY OF THE INVENTION

The present invention is a surface for medical and industrial objects that is made of a class of surfactants having a cyclic lipopeptide structure. Biofilm formation is an important medical and industrial problem and the ability to inhibit biofilm formation is an important application for surfactants. Surfactin, a cyclic lipopeptide surfactant, has the advantages of being able to be applied to surfaces prior to the formation of the biofilm and can impart long-term protection from biofilm formation.

In one embodiment, the present invention includes the use of lipopeptidic surfactants on the surface for the prevention of biofilm formation. The biosurfactant surfactin and its analogs may be used as such as a coating on the surface. One analog of surfactin is serrawettin. Surfactin and serrawettin can be used either singly, or in combination with various other substances to inhibit biofilm formation. Biofilm formation by organisms such as Escherichia coli, Proteus mirabilis, Salmonella typhimurium, Staphylococcus epidermis and Klebsiella pneumoniae can be inhibited by surfactin.

The surfactant coatings (either surfactin, serrawettin, or combinations of these with other substances), may be applied to a variety of objects of medical and industrial usage. The coating imparts resistance to biofilm formation on the object. These objects that may be coated include medical implants such as heart valves and catheters, wound care devices, personal protection devices, body cavity devices, and birth control devices. The method may also apply to the coating of teeth to prevent plaque formation, and to the coating of body piercings. Industrial objects may also be coated using these cyclic heptapeptides. Possible surfaces to be coated include water pipes, computer chips, and materials ranging from PVC to concrete.

Another embodiment of the present invention is a method of preventing biofilm formation by applying an effective protecting amount of the cyclic heptapeptides to that object. The method can be used to impart resistance to medical devices such as medical implants, wound care devices, personal protection devices, body cavity devices, and birth control devices. The method may also apply to coating of teeth, and to coating of body piercings. Industrial objects that may be coated include water pipes, computer chips, and materials ranging from PVC to concrete.

To be used in medical devices, the object that is coated would need to be at least partially sterilized and must withstand exposure to the aqueous solution in which the object is to be placed. Therefore, another embodiment of the present invention is a method of coating the objects wherein the coating process is followed by a heating step. Herein, the used heating refers to a treatment at 60° C. for at or about 1 hour or at 50° C. for at or about 6 hours).

### BRIEF DESCRIPTION OF THE DRAWINGS

For a more complete understanding of the features and further advantages of the present invention, reference is now made to the detailed description of the invention along with the accompanying FIGURES in which corresponding numerals in the different FIGURES refer to corresponding parts and in which:

FIG. 1 depicts kinetics of biofilm formation (BF) by wild-type Salmonella enterica (S. enterica) in accordance with the present invention;

FIG. 2 depicts surfactin inhibition of biofilm formation by wild-type S. enteria in accordance with the present invention;

FIG. 3 depicts dispersal of biofilm formation in accordance with the present invention;

FIG. 4 depicts biofilm formation in S. marcescens and its mutants in the presence of surfactin in accordance with the present invention; and

FIG. 5 depicts surfactin inhibition of biofilm formation on urethral catheters in accordance with the present invention.

### DETAILED DESCRIPTION OF THE INVENTION

Although the making and using of the various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention, and do not delimit the scope of the invention.

To facilitate the understanding of this invention, a number of terms are defined below. Terms defined herein have meanings as commonly understood by a person of ordinary skill in the areas relevant to the present invention. Terms such as “a,” “an,” and “the” are not intended to refer to only a singular entity, but include the general class of which a specific example is used for illustration. The terminology herein is used to describe specific embodiments of the invention, but their usage does not limit the invention, except as outlined in the claims.

All technical and scientific terms used herein have the same meaning as commonly understood by one of ordinary skill in the art to which this invention belongs, unless defined otherwise.

In nature, there is a prevalence of microbial colonies that remain attached to surfaces in associations also referred to as biofilms. Biofilms are composed of exopolysaccharides, a type of ‘slime’ that is secreted by the adherent bacteria. Bacteria that have formed adherent biofilms exist not as a tightly packed unit but rather as columns of loosely associated cells, some fixed, others motile. Water channels between pillars of cells in such biofilms allow nutrients to disperse. Motile colonies or colonies containing mobile bacteria are said to have swarming ability.

Biofilms are medically and industrially important because they can accumulate on a wide variety of substrates, disrupting the surface, altering its characteristics and often damage the substrate surface. More importantly, a growing population of organisms that create biofilms are becoming resistant to general use agents designed to remove them, such as antimicrobial agents and detergents. Therefore, inhibiting the initial microbial adhesion to surfaces is important.

The present invention includes adding an effective amount of surfactant to the surface of an object. This coating prevents the adhesion of microbes to the surface, and does not affect the viability of the microbe. Preserving the viability of microbes is attributable to the non-lethal nature of surfactin. Lethal compounds such as silver or antibiotics often create selective pressure to increase the likelihood of amplifying silver-resistant or antibiotic resistant strains, that eventually render the anti-biofilm agents useless. This is an important consideration when the object to be coated is a medical device that will be implanted in the body, where resident bacteria exist.

The apparatus and method of the present invention uses the cyclic lipopeptide surfactin to prevent biofilm formation. The biosurfactant surfactin is produced by and can be isolated from e.g., Bacillus subtilus. The effect of surfactin on biofilm formation by medically relevant organisms on microtitre plates, on vinyl urethral catheters and on central venous catheters made of polyurethane was investigated.

The ability of lipopolysaccharide (LPS) mutants to form biofilms was tested in PVC microtitre plates. The biofilm assay used monitors the ability of S. enterica to attach to the wells of the microtitre dishes. The biofilm formed at the interface between the air and liquid medium, and was quantitated by staining with crystal violet (CV) as described in the examples given below. Initial studies with different abiotic materials (PVC, polystyrene, borosilicate glass) showed that the wild-type strain SJW1103 forms the best biofilms on PVC in Luria-Bertani broth (LB) without sodium chloride (NaCl) but with 0.2% glucose, and at 30° C.

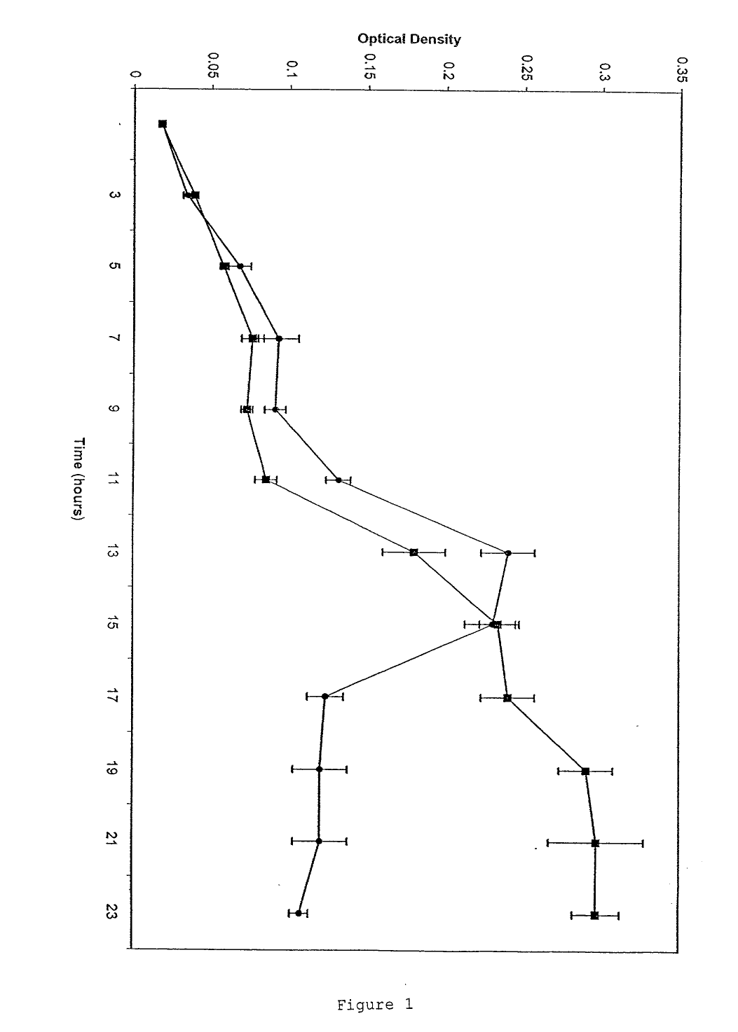

FIG. 1 shows the kinetics of biofilm formation (BF) by wild-type S. enterica. The exponential phase of BF coincided with that of cell growth. BF began to slow down at around 13 hours and decreased up to 17 hours, and then leveled off, coincident with the entry of the culture into stationary phase.

Studies were done to test biofilm formation in microtitre wells. To quantify biofilm formation, typically, 10 μl of an overnight culture were used to inoculate PVC microtitre wells containing 90 μl of LB without NaCl, but with 2% glucose. The covered microtitre dish was sealed with parafilm during incubation at 30° C. Cultures were removed to determine the OD₆₃₀, and the wells were rinsed with distilled water. After drying at room temperature for 15 minutes, 200 μl of crystal violet (1%) was added to the wells for 20 minutes The stained biofilms were rinsed several times with distilled water, allowed to dry at room temperature for 15 minutes, and extracted with 2×200 μl 95% ethanol. The OD₅₅₀ was estimated using a Beckman DU-640B spectrophotometer, after adjusting the volume to 1 mL with distilled water.

The swarming defect of the LPS mutants could be rescued by the addition of the surfactin isolated from Bacillus subtilis. This led to the investigation of whether surfactin could inhibit biofilm formation by S. enterica. To analyze the effect of surfactin on BF, the PVC wells were either pre-coated with surfactin, or surfactin was included in the growth medium. In these studies, PVC coated wells were coated prior to inoculating with S. enterica and incubating overnight at 30° C. The wells were rinsed out and stained with crystal violet.

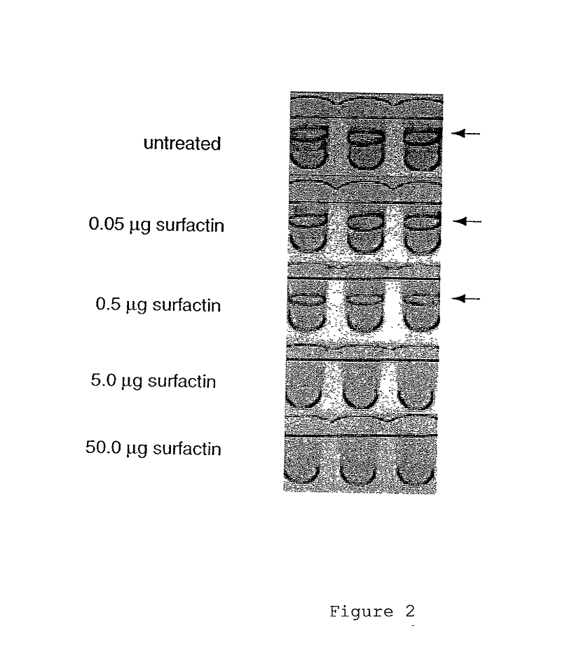

FIG. 2 shows that the biofilm was concentrated at the interface between the air and liquid medium. Increasing amounts of surfactin led to a decrease in the amount of biofilm formed by the wild-type S. enterica and 5 μg of surfactin was more than sufficient to completely abolish BF. Bacterial growth was unaffected under all surfactin concentrations tested, an important consideration for practical applications such as the coating of medical devices.

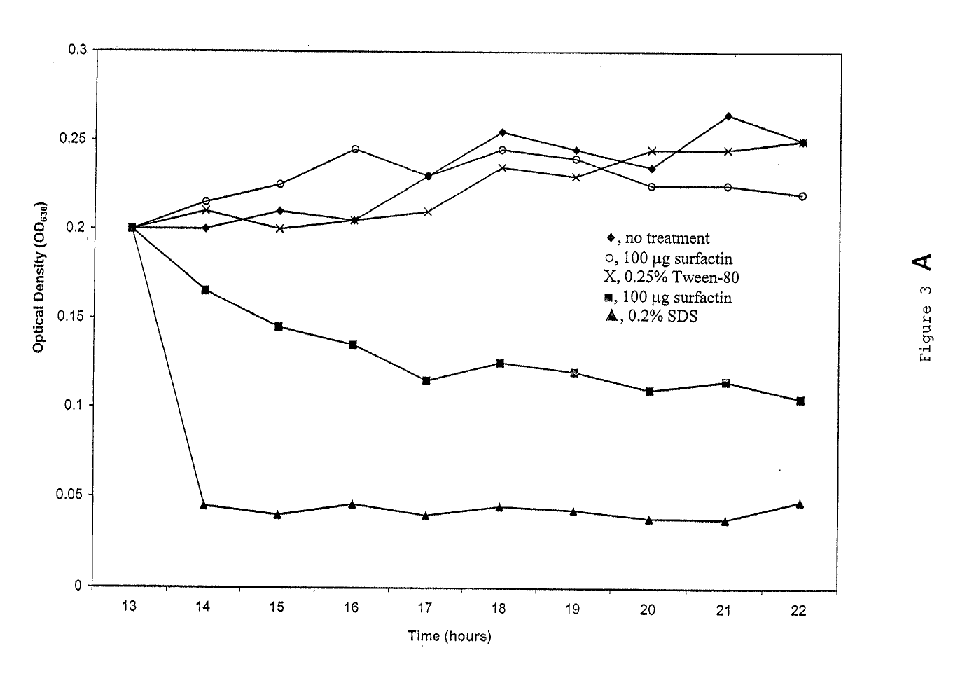

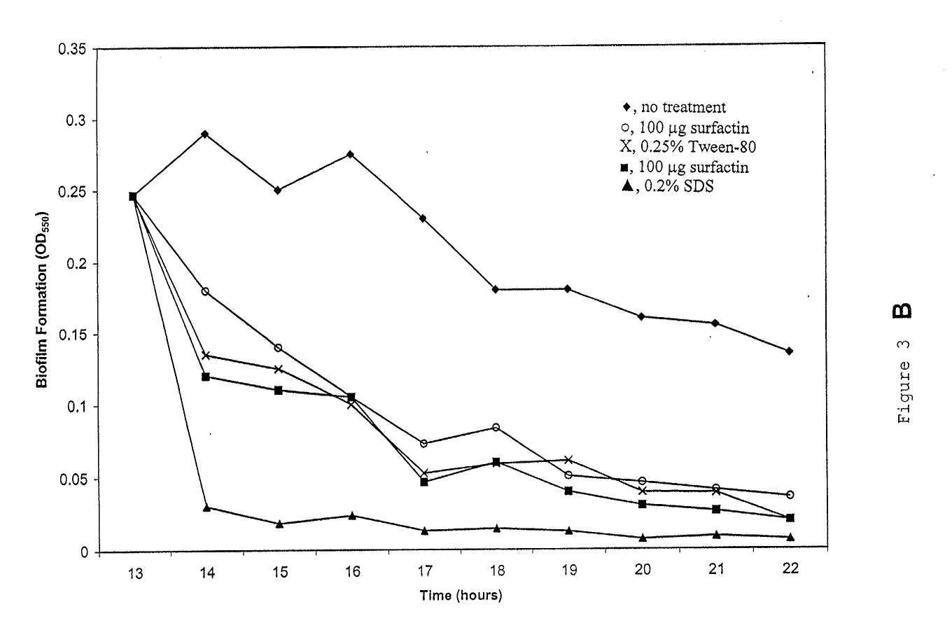

FIG. 3 shows the determination of whether surfactin would dislodge a pre-formed biofilm. Surfactin was added to PVC wells after the culture had reached an OD₆₃₀ of approximately 0.15-0.2. When this OD was reached, the surfactants were gently mixed into the cultures in microtitre wells. Samples were harvested and either growth as determined by OD₆₃₀ or biofilm levels as measured by OD₅₅₀ of CV-stained material were analyzed. The OD₅₅₀ of the surfactin-treated sample decreased at a faster rate than that of the untreated sample for the initial sloughing phase of BF, resulting in an approximately 85% decrease in total biofilm by the end of the experiment at 22 hours.

FIG. 3 shows the effect of a variety of detergent-like compounds on pre-formed biofilms. The detergents tested were SDS (ionic surfactant), Tween-80 (anionic surfactant), rhamnolipid (another lipopeptide surfactant) and serrawettin. Surfactin concentration in this and the rest of the studies was maintained at 100 μg in order to compare its activity to that of the biosurfactant rhamnolipid, which affected BF when it was used at higher concentrations. All of the tested chemicals dispersed pre-formed biofilm.

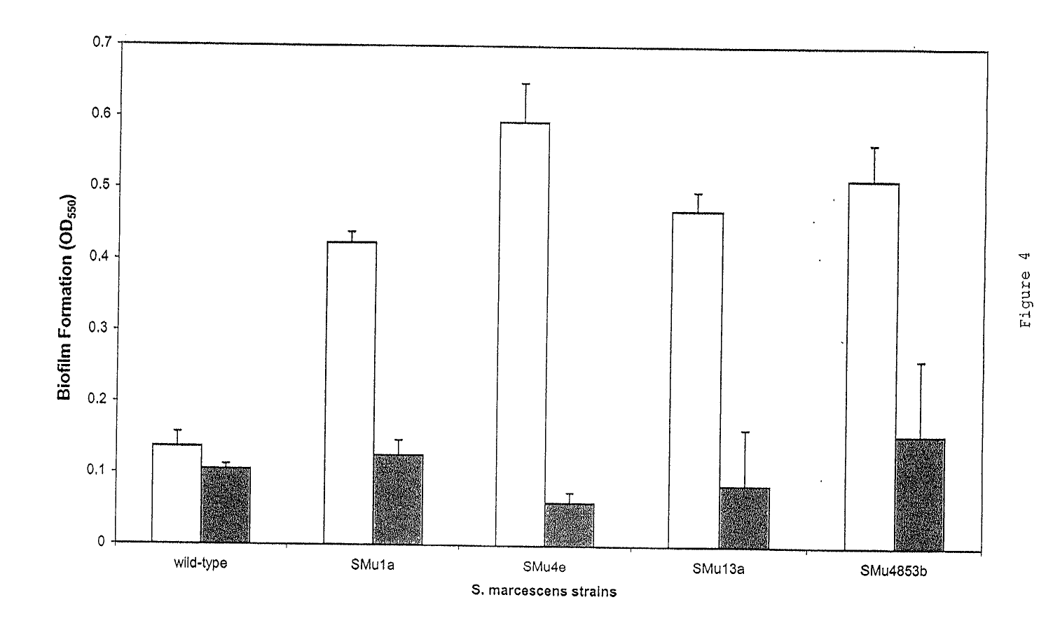

FIG. 4 shows the biofilm-forming ability of bacteria known to produce surfactants. Both wild-type and mutant strains of S. marcescens and B. subtilis were investiagted. In S. marcescens, mutants defective in the production of the surfactant serrawettin are unable to swarm, as are surfactant mutants of B. subtilis. Mutants of S. marcescens that were defective in serrawettin made approximately three-fold more biofilm than their wild-type counterparts. These results are consistent with the notion that the absence of the biosurfactant promotes biofilm formation.

To visualize biofilm formation in catheters, 10 μl of an overnight culture of S. enterica was inoculated into 500 μl of medium and injected into clear vinyl urethral catheters overnight at 30° C., with and without 100 μg surfactin. Biofilms were analyzed by staining with CV. The catheters were capped at both ends and incubated at 30° C. overnight. Media and growth conditions were as described above for PVC wells. Cultures were removed to determine the OD₆₃₀, and the catheters were rinsed with distilled water. After drying at room temperature for 15 minutes, 700 μl of crystal violet (1%) was added to the catheters for 20 minutes. The stained biofilms were rinsed several times with distilled water, and allowed to dry at room temperature for 15 minutes before examination.

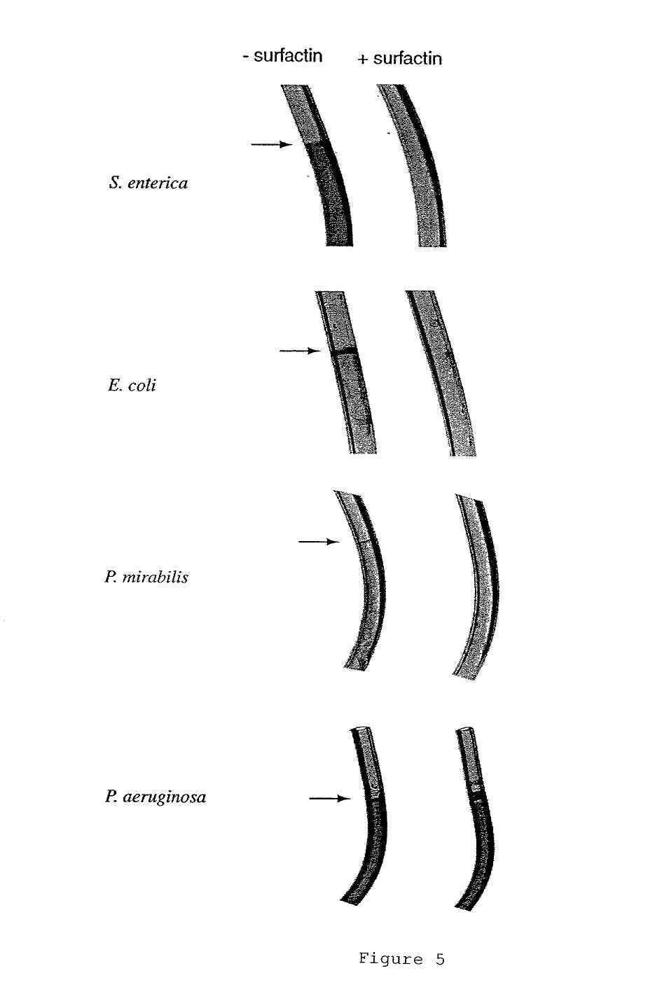

FIG. 5 shows the effect of the surfactin on medically relevant objects. S. enterica was grown in clear vinyl urethral catheters. The biofilm formed by S. enterica was dispersed all along the growth surface. Surfactin eliminated the formation of biofilm on the catheters (Table 1). It is important to note that the same results were obtained when venous catheters made of polyurethane were tested. The data presented here relate mainly to the urethral catheters.

When the device coated is to be inserted in the body cavity, some form of surface sterilization may be necessary. Also, endogenous fluids should not wash off the surfactin coating. Studies were conducted to determine these properties of the coating (Table 1). Urethral catheters were coated with surfactin (by passing through 500 μl of a solution of 1.0 μg/μl surfactin), and 10 mL of sterile saline solution were passed through the coated catheter. This washing step was found to remove surfactin from the catheter allowing Salmonella typhimurium biofilm to form.

After coating urethral cathethers with surfactin, the coated catheters were subjected to treatment in an autoclave (121° C., 15 psi) for 30 minutes or baking in a 50° C. oven for 6 hours. Autoclave treatment reduced the biofilm-inhibiting efficacy of surfactin by approximately 40%, but oven treatment had no effect on biofilm formation by surfactin. Additionally, it was observed that oven treatment of surfactin coated catheters “baked” surfactin onto the catheters rendering them resistant to saline washing. Surfactin, apparently adhered to the catheters, largely inhibiting biofilm formation.

TABLE 1
Effect of various catheter treatments on biofilm
formation by Salmonella typhimurium.

| Catheter treatment | Biofilm Formation^{a} |
|---|---|
| Untreated | ++++ |
| Surfactin | − |
| Surfactin, saline wash | ++++ |
| Surfactin, autoclave | ++ |
| Surfactin, the oven | − |
| Surfactin, then oven then saline wash | + |

^{a}“++++” = efficient biofilm formation; “−” = no biofilm formation.

The biofilm-inhibiting properties of surfactin are not altered after storing surfactin-baked catheters (baked for one hour at 60° C.) for 5 days at room temperature (Table 2). Further, baked on surfactin is not washed off by sterile saline dripping through the catheter at 0.3 mL/minutes for 24 hours. The BF-inhibiting properties of surfactin are stable over 50 days of storage at either room temperature or at 4° C. Thus, medical devices coated with surfactin, or a substance with surfactin-like properties, may be partially sterilized by baking at 60° C., and the sterility would be maintained over a long period of time. Also, the 40% reduction after autoclaving (as seen in Table 1) may not be significant when there are smaller numbers of bacteria present (i.e., bacteria concentrations used in these studies are on the order of a million times greater than those encountering medical devices).

TABLE 2
Biofilm formation on catheters coated with surfactin
and subjected to various treatments. The numbers are an
optical density reading based on crystal violet staining.

| Organism | 5 days at room T. | 24 hour saline wash |
|---|---|---|
| Salmonella | 0.05 | 0.06 |
| Typhimurium | | |

Pre-coating catheters by running the surfactin solution through them prior to inoculation with medium was just as effective as including surfactin in the growth medium. Among other surfactants tested for inhibition of BF by S. enterica, Tween® 80 (0.25%) was as effective as surfactin, while rhamnolipid seemed only half as effective. It is important to note, however, that these assays were done with between 10 and 100 million bacterial cells. In a hospital setting, the patient's catheters will be exposed to far fewer bacteria. Hence, rhamnolipid may function as effectively in this capacity as surfactin. Given the opportunistic infections with Salmonella species, including central urinary catheter tract infections of AIDS patients, these results have the potential for practical applications.

The most common causes of central urinary catheter and central venous catheter infections (caused by adherent bacteria), include Eschericila coli, Proteus mirabilis, and Pseudomonas aeruginosa, Klebseiella pneumoniae, Staphylococcus epidermis. The effect of surfactin on BF by some of these medically relevant organisms was tested by growth of the organism in urethral catheters (Table 3). Escherichia coli and Proteus mirabilis formed a biofilm mainly at the air liquid interface, while the biofilm formed by P. aeruginosa, like that formed by S. enterica, was dispersed all along the catheter. Surfactin inhibited BF (but not growth) in all organisms except P. aeruginosa.

TABLE 3
Biofilm formation by various bacteria on surfactin-
treated and uncoated catheters. The number are an optical
density reading based on crystal violet staining.

| Organism | Surfactin-coated | Uncoated |
|---|---|---|
| Salmonella typhimurium | 0.05 | 0.81 |
| Escherichia coli | 0.05 | 1.05 |
| Proteus mirabilis | 0.11 | 0.89 |
| Staphylococcus epidermis | 0.40 | 2.20 |

Given the effectiveness that surfactin, and some related chemicals that were tested had on dissipating pre-formed biofilm and on preventing biofilm formation, there are numerous applications in addition to both venous and urethral catheters. The use of surfactin as a surface coating for a variety of materials is one such application. However, other variations are possible. For example, surfactin can be mixed with liquids such as paint and molten plastic. In this way, the anti-biofilm properties are imparted by incorporating them directly into the material versus the direct coating of the object with the surfactin.

While the invention has been described in reference to illustrative embodiments, the description is not intended to be construed in a limiting sense. Various modifications and combinations of the illustrative embodiments, as well as other embodiments of the invention, will be apparent to persons skilled in the art upon reference to the description. It is therefore intended that the appended claims encompass any such modifications or embodiments.

What is claimed:

1. A coating for surfaces comprising one or more lipopeptides that inhibit biofilm formation.

2. The coating recited in claim 1 wherein the effective amount of lipopeptides is in the range of 5 to 100 μg.

3. The coating recited in claim 1 wherein the concentration of lipopeptides in the solution ranges from 0.1 to 5.0 μg/μl.

4. The coating recited in claim 1 wherein the lipopeptide further comprises one or more of the group consisting of a cyclic lipopeptide, cyclic heptapeptide, surfactin, serrawettin, and analogs and derivatives of surfactin and serrawettin.

5. The coating recited in claim 4 wherein the lipopeptide is in combination with other chemicals.

6. A coating for medical devices that prevents formation of a biofilm comprising a lipopeptide coated on the surface and a medical device having a surface.

7. The coating recited in claim 6 wherein the lipopeptide further comprises one or more of the group consisting of a cyclic lipopeptide, cyclic heptapeptide, surfactin, serrawettin, and analogs and derivatives of surfactin and serrawettin.

8. The coating recited in claim 7 wherein the lipopeptide is in combination with other chemicals.

9. The coating recited in claim 6 wherein the medical device is selected from the group consisting of contact lens, medical implant, wound care device, personal protection device, body cavity device, birth control device, heart valve, catheter.

10. The coating recited in claim 9 wherein the catheter further comprises one or more of the group consisting of a urethral catheter and central venous catheter.

11. A coating for industrial devices that prevents formation of a biofilm comprising a lipopeptide and an object with a surface.

12. The coating recited in claim 11 wherein the lipopeptide further comprises one or more of the group consisting of a cyclic lipopeptide, cyclic heptapeptide, surfactin, serrawettin, and analogs and derivatives of surfactin and serrawettin.

13. The coating recited in claim 12 wherein the lipopeptide is in combination with other chemicals.

14. The coating recited in claim 11 wherein the object is selected from the group consisting of computer chip, water pipe, metal, plastic, concrete, glass, stainless steel, acrylic, polyvinylchloride, polyurethane, and silicone.

15. The coating recited in claim 11 wherein the object is a body piercing.

16. A coating comprising a lipopeptide and a surface to be coated wherein the surface to be coated is teeth.

17. A paint that prevents biofilm formation comprising paint and a lipopeptide mixed with the paint.

18. The paint recited in claim 16 wherein the lipopeptide further comprises one or more of the group consisting of a cyclic lipopeptide, cyclic heptapeptide, surfactin, serrawettin, and analogs and derivatives of surfactin and serrawettin.

19. The paint recited in claim 18 wherein the lipopeptide is in combination with other chemicals.

20. A method of constructing plastic that prevents biofilm formation comprising the steps of:
using molten plastic; and
mixing lipopeptide with the molten plastic.

21. The method recited in claim 20 further comprising the step of pouring the mixture of the molten plastic and the lipopeptide into a mould.

22. The method recited in claim 20 wherein the lipopeptide is selected from the group consisting of a cyclic lipopeptide, cyclic heptapeptide, surfactin, serrawettin, and analogs and derivatives of surfactin and serrawettin.

23. The method recited in claim 22 wherein the lipopeptide is in combination with other chemicals.

24. A method of imparting protection against biofilm formation to an object comprising:
applying an effective amount of a lipopeptidic surfactant to the object.

25. The method recited in claim 24 wherein the effective amount of the lipopeptidic surfactant is in the range of 5 to 100 μg.

26. The method recited in claim 24 wherein the lipopeptidic surfactant is selected from the group consisting of a cyclic lipopeptide, cyclic heptapeptide, surfactin, serrawettin, and analogs and derivatives of surfactin and serrawettin.

27. The method recited in claim 26 wherein the lipopeptidic surfactant is in combination with other chemicals.

28. The method recited in claim 24 further comprising:
passing an object through of a solution of lipopeptidic surfactant; and
baking the object at 60° C. for 1 hour.

29. The method recited in claim 28 wherein the lipopeptidic surfactant is surfactin in a solution concentration range from 0.1 to 5.0 μg/μl.

30. The method recited in claim 24 wherein the object is selected from the group consisting of a medical device, contact lens, medical implant, wound care device, personal protection device, body cavity device, birth control device, heart valve, catheter, urethral catheter, central venous catheter catheter, and a body piercing.

31. The method recited in claim 24 wherein the object has an industrial use.

32. The method recited in claim 24 wherein the object further comprises one of a group consisting of a computer chip, synthetic material, natural material, water pipe, metal, plastic, concrete, glass, stainless steel, acrylic, polyurethane, silicone, polyvinylchloride.

33. A method of dissipating biofilm formation comprising:
addition of surfactin to the biofilm.

34. The method recited in claim 33 wherein the biofilm is in an aqueous system.

35. The method recited in claim 33 wherein the biofilm is on a surface.

36. The method recited in claim 35 wherein the surface is selected from the group consisting of medical device, industrial device, metal, acrylic, stainless steel, glass, teeth, polyvinylchloride, and a computer chip.
